(19) United States
(12) Patent Application Publication
(10) Pub. No.: US 2004/0110196 A1
(43) Pub. Date: Jun. 10, 2004

(54) Method and composition for forming a protective coating on an assay substrate and substrate produced by the same

(76) Inventors: James M. Kadushin, Gilbertsville, PA (US); Robert C. Getts, Collegeville, PA (US)

Correspondence Address: WATOV & KIPNES,P.C., P.O. Box 247, Princeton Junction, NJ, 08550, US

(21) Appl. No.: 10/643,322

(22) Filed: Aug. 19, 2003

Related U.S. Application Data
(60) Provisional application No. 60/406,015, filed on Aug. 26, 2002.
(60) Provisional application No. 60/404,958, filed on Aug. 21, 2002.

(51) Int. Cl. C12Q 1/68; G01N 33/53; C12M 1/34

(52) U.S. Cl. 435/6; 435/7.1; 435/287.2

(57) ABSTRACT

A composition for forming a protective coating over the surface of an assay substrate having an indicating agent capable of generating a detectable signal associated therewith, includes a protective coating forming material, and a delivery system for delivering the protective coating forming material in an amount sufficient to coat the surface of the assay substrate, wherein the delivery system evaporates from the surface of the assay substrate to form a protective coating at least substantially composed of the protective coating forming material that is at least substantially transparent to the detectable signal generated by the indicating agent. The present invention is further directed to a method for forming the protective coating over the surface of the assay substrate, and a substrate produced by such method.

### RELATED APPLICATIONS

The present Application claims the priority of U.S. Provisional Application Serial No. 60/406,015, filed Aug. 26, 2002, and entitled “METHOD AND DEVICE FOR PRESERVING THE FLUORESCENCE OF FLUOROCHROMES FROM DEGRADATION IN TYPICAL LABELING/DETECTION ASSAYS BY USING A NON-FLUORESCENT TRANSPARENT COATING”, and further claims the priority of U.S. Provisional Application Serial No. 60/404,958, filed August 21, 2002, and entitled “METHOD AND DEVICE FOR PRESERVING THE FLUORESCENCE OF FLUOROCHROMES FROM DEGRADATION IN TYPICAL LABELING/DETECTION ASSAYS BY USING A NON-FLUORESCENT TRANSPARENT COATING”, the contents of which are fully incorporated herein by reference

### FIELD OF THE INVENTION

The present invention relates to methods and compositions for forming a protective coating on an assay substrate, and more particularly to methods and compositions for forming a protective coating to extend the life and slow the rate of oxidation of signal generating dyes and labels including fluorescent molecules used in association with assay substrates.

### BACKGROUND OF THE INVENTION

Assays including, for example, DNA microarrays, utilize a range of indicating agents typically in the form of labels and dyes for revealing the qualitative and/or quantitative characteristics (i.e., presence, absence, measure or quality) of one or more components in a sample. These indicating agents typically generate information or signal that is thereafter detected and analyzed to make the corresponding examination and determination. The signal generation of the chemically active indicating agents is generally initiated through chemical reactions or events triggered by the presence or absence of the component(s) of interest. Since these indicating agents are chemically active, the agents are frequently sensitive to the presence of oxidizers such as ozone and other substances commonly present in ambient air.

For example, traditional nucleic acid detection is typically performed by hybridizing two complementary strands of nucleic acid (DNA or RNA), a target and a probe, each of which having labeled nucleotides incorporated directly or indirectly into the strand to generate a detectable signal. The label may be a radioisotope such as ³²P, biotin, digoxigenin, various fluorescent molecules, visible dyes, contrast agents including enzyme-based contrast agents as well as others known in the art. Each type of label requires a unique development and detection scheme to generate positive results. Depending on the type of assay, one label may be preferred over another, and the proper selection of a suitable label is known to one experienced in the art. In an embodiment of the present invention, the labels of special interest are fluorochromes or fluorescent dyes. These labels are typically incorporated directly as nucleotide analogs, as in nucleic acid based assays or as chemical derivatives as in protein based assays.

Some common examples of typical assays include fluorescent in situ hybridization (FISH), immunohistochemical assays, and fluorescent microarrays used for gene expression detection and analysis. Once the fluorescent label has been integrated into the assay as a reporter, it is detected using some type of instrumentation specifically designed for the assay, the selection of which is well known to those experienced in the art. Common to all of the instruments designed to measure fluorescence is a light source, such as a laser, capable of emitting light at a wavelength, referred to as the “excitation wavelength”, suitable to ‘excite’ electrons in the fluorochrome atom to an alternate energy level. The electron then reverts to the original energy level emitting a photon of energy. This property is commonly referred to as fluorescence. The released energy is typically at a longer wavelength (lower energy) as compared to that required to initially excite the fluorochrome. The emitted energy is detected by the instrument and converted to a suitable output format. Examples of common fluorochromes or fluorescent dyes include the Cyanine™ dyes, Cy3 and Cy5, of Amersham Pharmacia Biotech Inc. of Piscataway, N.J., and the Alexa Fluor™ dyes, Alexa 488, Alexa 546, Alexa 594 and Alexa 647, and the like, each available from Molecular Probes.

Unlike radioactive labels, fluorescent molecules do not elucidate a natural decay process. However, as the inventors of the present invention have discovered, fluorescent labels are sensitive to agents that may change their chemical structure thus diminishing or destroying their fluorescent properties. Examples of such fluorescent label affecting agents include light, oxidative agents found in air such as ozone, basic compounds, and the like. In some cases these agents may generate free radicals that will derivatize or alter the chemical structure of the fluorochrome. The ‘red channel’ dyes, Cy5, Alexa 647, and the like, are most susceptible to this form of degradation and often data from experiments including these dyes is unusable and the experiment must be repeated. Also, in many cases the signal from these ‘red channel’ dyes is short lived and can degrade to less than 10% of the starting signal in as little as 1-5 minutes after completion of the assay. It is next to impossible to obtain reliable results if a prolonged period of time passes prior to signal detection on the reading instrument.

One example of an assay commonly prone to unreliable results due to degradation of the fluorescent signal is gene expression analysis on microarrays. Generally, a microarray comprises a support means having a substantially planar substrate such as a glass microscope slide, a silicon plate or nylon membrane, coated with a grid of tiny spots or features of about 20 microns in diameter. Each spot or feature contains millions of copies of a specific sequence of nucleic acid extracted from a strand of deoxyribonucleic acid (DNA). Due to the number of features involved, a computer is typically used to keep track of each sequence located at each predetermined feature. Each microarray is capable of performing the equivalent of thousands of individual “test tube” experiments over a short time period thereby providing rapid and simultaneous detection of thousands of expressed genes. Microarrays have been implemented in a range of applications such as analyzing a sample for the presence of gene variations or mutations (i.e. genotyping), or for gene expression profiling.

For expression analysis, messenger RNA (mRNA) is extracted from a sample of cells. The mRNA, serving as a template, is typically reverse transcribed to yield a complementary DNA (cDNA). As a first example of the prior art techniques, one or more indicating agents or detection means, including labels or markers such as fluorescent dyes, are directly incorporated into the copies of cDNA during the reverse transcription process. Under suitable hybridization conditions, the labeled fragments are hybridized or coupled with complementary nucleic acid sequences (i.e. gene probes) attached to the features of the microarray for ready detection thereof. This labeling method has been commonly referred to as “direct incorporation”. Alternatively, other labeling methods may also be used in the present invention, such as, for example, labeling methods utilizing dendrimers as described in U.S. Pat. Nos. 5,175,270; 5,484,904 and 5,487,973, the contents of which are incorporated herein by reference, or labeling methods utilizing linear polymers. Linear polymers are described in U.S. Provisional Patent Application No. 60/388,196, the content of which is incorporated herein by reference.

Upon hybridization of the cDNA to the microarray, a detectable signal (e.g. fluorescence) is emitted for a positive outcome from each feature containing a cDNA fragment hybridized with a complementary gene probe attached thereto. The detectable signal is visible to an appropriate scanner device or microscope, and may then be analyzed by the computer or user to generate a hybridization pattern. Since the nucleic acid sequence at each feature on the microarray (the probe) is known, any positive outcome (i.e. signal generation) at a particular feature indicates the presence of the complementary cDNA sequence in the sample cell. Although there are occasional mismatches, the attachment of millions of gene probes at each spot or feature ensures that the detectable signal is strongly emitted only if the complementary cDNA of the test sample is present. However, if the signal of a particular assay is prone to rapid degradation, the results are of little use and can be difficult to interpret.

It would be desirable to develop a composition and method for forming a protective coating on an assay substrate to protect and preserve the indicating agents (e.g. fluorescent dyes or labels) from environmental agents such as ozone, dust and the like which can adversely affect the same. There is a need to provide a protective coating that hermetically seals the indicating agents associated with the assay substrate and isolates it from the ambient atmosphere to prolong the life of the indicating agents and to provide substantial transparency to facilitate passage of the signal generated by the indicating agent while minimally contributing an adverse amount of nonspecific signal generation (i.e., background noise), which could undesirably interfere with the detection of the signal generated by the indicating agent.

### SUMMARY OF THE INVENTION

The present invention is generally directed to a composition and method for forming a protective coating over the surface of an assay substrate having an indicating agent capable of generating a detectable signal associated therewith. The composition includes a delivery system for delivering a protective coating forming material to the surface of the assay substrate. The delivery system is formulated to evaporate in a controlled manner leaving behind the protective coating on the surface of the assay substrate. The resulting protective coating formed from the composition and method of the present invention hermetically seals the indicating agent from ambient atmosphere, thereby significantly extending the signal generating life of the indicating agent. The protective coating further provides substantial transparency to the signal generated by the indicating agent, while avoiding or minimizing nonspecific signal generation (i.e., background noise), which could undesirably interfere with the detection of the signal generated by the indicating agent. The present invention is further directed to an assay substrate produced by the present methods and compositions.

In a particular aspect of the present invention, there is provided a composition for forming a protective coating over the surface of an assay substrate having an indicating agent capable of generating a detectable signal associated therewith, comprises a protective coating forming material, and a delivery system for delivering the protective coating forming material in an amount sufficient to coat the surface of the assay substrate, wherein the delivery system evaporates from the surface of the assay substrate to form a protective coating at least substantially composed of the protective coating forming material that is at least substantially transparent to the detectable signal generated by the indicating agent. The composition of the present invention may be in the form of a liquid or an aerosol.

In another aspect of the present invention, there is provided a method for forming a substantially transparent protective coating over the surface of an assay substrate having an indicating agent capable of generating a detectable signal associated therewith, the method comprises the steps of:

applying to the surface of the assay substrate an effective amount of the composition described above sufficient to form the protective coating and to seal the indicating agent from the ambient atmosphere; and

drying the composition to remove the delivery system to yield the substantially transparent protective coating.

In a further aspect of the present invention, there is provided a substrate suitable for use in an assay comprising:

(a) a material for supporting an indicating agent;

(b) an indicating agent capable of generating a detectable signal; and

(c) a protective coating comprised principally of a protective coating forming material at least substantially transparent to the detectable signal generated by the indicating agent.

### BRIEF DESCRIPTION OF THE DRAWINGS

The following drawings are illustrative of embodiments of the present invention and are not intended to limit the invention as encompassed by the claims forming part of the application.

FIG. 1 represents an image of one of the two microarrays treated with the composition of the present invention and a second untreated microarray; and

FIG. 2 represents an image of a microarray treated with the composition of the present invention and a second untreated microarray.

### DETAILED DESCRIPTION OF THE INVENTION

The present invention is generally directed to a composition and method developed for forming a protective coating over the surface of an assay substrate having an indicating agent capable of generating a detectable signal associated therewith, in a manner that hermetically seals the indicating agent from ambient atmosphere to significantly extend the life of signal generated therefrom, while providing substantial transparency to permit passage of the detectable signal therethrough. The protective coating formed from the composition of the present invention is further imparted with the advantage of avoiding the generation of an adverse amount of nonspecific signal generation that would otherwise obscure or make difficult the detection of the signal generated by the indicating agent. The composition of the present invention may be in the form of a liquid or an aerosol. The present invention is further directed to an assay substrate produced by the present methods and compositions.

The composition of the present invention is compatible for use with a wide variety of substrates including glass substrates, aminosilane substrates, epoxy substrates, poly-L-lysine substrates and the like. The indicating agents include common fluorochromes or fluorescent dyes, radioisotopes such as ³²P, biotin, digoxigenin, various fluorescent molecules, visible dyes, contrast agents including enzyme-based contrast agents as well as others known in the art. The indicating agents may be used by direct incorporation, amino allyl and the like.

In one embodiment of the present invention, the present composition includes a delivery system for delivering a protective coating forming material to the surface of an assay substrate. The delivery system may be composed of an aqueous based solvent system or an organic solvent based system depending on the particular type of protective coating forming material capable of being solvated and delivered. The protective coating forming material is preferably of the type that is capable of forming a protective coating composed of a pure, medium-hard thermoplastic acrylic resin that is age resistant, non-crosslinking and at least substantially transparent upon casting. In one preferred embodiment, the protective coating forming material is an acrylic polymer resin selected, for example, from copolymers of methyl acrylate and ethyl methacrylate, and the like. Some representative examples of suitable acrylic resins are Acryloid AT-410, Paraloid® B72 and Paraloid® B66, each of which are commercially available from Rohm & Haas.

In one embodiment of the present invention, present composition includes the protective coating forming material in the amount of from about 1% to 90% by volume based on the total volume of the composition, more preferably from about 10% to 50% by volume, and most preferably at about 9% by volume.

The delivery system of the present invention is formulated to readily form a solution with the protective coating forming material of the present invention, and to evaporate therefrom in a controlled manner thereby leaving behind a protective coating composed at least substantially of the protective coating forming material, preferably at least 95%, most preferably at least 98% and desirably approaching 100%. The resulting protective coating exhibits the desired properties described hereinafter.

In one embodiment of the present invention, the delivery system is composed of at least one evaporative solvent capable of solvating the protective coating forming material and capable of readily evaporating in a controlled manner upon application of the composition to the surface of the assay substrate. Suitable evaporative solvents include organic and aqueous solvents depending on the protective coating forming material used. Examples of such solvents include, but are not limited, to water, alcohols, toluene, xylene, heptane, butanol, n-butyl acetate, methyl amyl ketone, ethyl benzene, ethylene glycol butylether, ethyl-3-ethoxypropionate, isopropyl alcohol, Aromatic 150, ethanol, methoxypropanol, acetone and combinations thereof, and the like.

The protective coating deposited by the delivery system exhibits good transparency across excitation wavelengths necessary for exciting certain indicating agents such as fluorescence dyes or fluorochromes, and to the fluorescence wavelengths generated therefrom to permit detection. The protective coating is sufficiently impervious to the penetration of gases including atmospheric gases such as ozone, for example. In this manner, the protective coating provides an effective barrier against external environment agents that are capable of undesirably degrading, oxidizing or reducing the integrity of the indicating agents. Further, the protective coating does not generate adverse amounts of nonspecific signals (i.e., background noise) in the form of fluorescence, which may interfere with the detection of the signals generated by the indicating agents.

The composition of the present invention may further include preserving agents, which act to minimize or eliminate yellowing or discoloration of the protective coating due to oxidative or other adverse effects during the evaporation of the delivery system and during the life of resulting protective coating. The incorporation of the optional preserving agents significantly enhances signal transmissibility and detection, while further reducing or eliminating undesirable nonspecific signal generation or fluorescence. Such preserving agents may be selected from aromatic naphthas such as naphthalene, and the like. The amount of the preserving agent incorporated into the compositions of the present invention may range from about 0.1% to 10% by volume based on the total volume of the composition, more preferably from about 0.2% to 1.5% by volume, and most preferably at about 0.52% by volume.

In one embodiment of the present invention, the composition of the present invention includes a copolymer of methyl acrylate and ethyl methacrylate, in combination with a delivery system composed of acetone, toluene, naphthalene, and ethyl-3-ethoxypropionate. In one preferred embodiment, the composition comprises 8.75% by volume copolymer of methyl acrylate and ethyl methacrylate, 0.52% by volume naphthalene, 26.35% by volume acetone, 64.03% by volume toluene, and 0.35% by volume ethyl-3-ethoxypropionate, each of which are based on the total volume of the composition.

The composition of the present invention may further include other petroleum distillates, which may be normally present in trace amounts with the acrylic polymer resins. The aerosol form of the present compositions may further contain hydrocarbon propellants and the like.

The present invention is further directed to methods for forming a substantially transparent protective coating over the surface of an assay substrate having an indicating agent capable of generating a detectable signal associated therewith. A prepared assay substrate with the test material and indicating agent is first dried to remove excess moisture. The assay substrate is preferably maintained in a clean dust free condition. If necessary, the assay substrate may be blowed with compressed air to remove any potential contaminants or environmental agents, which may degrade the indicating agent. The composition of the present invention is applied to the surface of the assay substrate in contact with the indicating agent. The application of the present composition on the assay substrate can be made via any suitable techniques including spraying, dipping, pipetting, and the like. Preferably, the assay substrate is dipped into a bath containing the present composition. The assay substrate is submerged from about 5 seconds to 10 seconds. The assay substrate is withdrawn from the bath and any excess is blotted or drawn off to a towel. The assay substrate is positioned vertically and allowed to dry from about 3 minutes to 5 minutes.

Optionally, the steps for applying the present composition may be repeated after drying to further enhance the protection of the indicating agent against the adverse effects of exposure to ambient air.

Once the protective coating forming material is dried, the coated assay substrate may be scanned for detection and analysis. The coated assay substrate may be stored in a dark area and the indicating agent may remain viable for at least a period of 3 weeks.

In an alternative embodiment, the composition of the present invention may be applied to the assay substrate by pipetting a suitable amount across the entire surface of the assay substrate. The assay substrate is then rocked side to side for less than 3 seconds to evenly distribute the composition across the assay substrate surface. Thereafter, position the assay vertically to allow any excess composition of the present invention to be drawn off onto a towel. Allow the assay substrate to dry in the vertical position from about 3 minutes to 5 minutes.

Optionally, once the protective coating forming material is dried, the assay substrate may be treated for further processing to enhance signal scans or readings. The protective coating may be unevenly distributed thus possibly creating signal artifacts or distortions that may adversely affect the scan and results of the assay. The uneven distribution of the protective coating may be corrected by implementing a polishing procedure. The polishing procedure may be repeated as necessary to remove the uneven distribution. In one embodiment of the present invention, a polishing solution is prepared containing a solvent mixture. The solvent mixture comprises at least one suitable solvent capable of solvating and removing at least a layer portion of the dried protective coating from the assay substrate without adversely affecting the remaining portions of the protective coating. Preferably, the solvent mixture comprises a mixture of acetone and toluene in a volumetric ratio of from about 1:3 to 1:2. In a preferred embodiment, the solvent mixture contains acetone and toluene in a volumetric ratio of about 1:3.

A bath containing the polishing solution is prepared. The assay substrate is immersed into the polishing solution and quickly withdrawn there from. The polishing solvent removes layer portions of the dried protective coating on the substrate surface. The assay substrate is allowed to dry in the upright position. The polishing procedure may be repeated as necessary until a more even distribution of the protective coating is produced.

### EXAMPLE 1

Although reference will generally be made herein to microarrays for illustration purposes, it is to be understood that the invention is not limited to microarrays, but may be used with blots or any other assay formats currently in use or later developed in the art that include detection means, such as fluorescent dyes or the like, that are sensitive to agents that may change their chemical structure thus diminishing or destroying their ability to be detected, i.e. in the case of fluorescent dyes their fluorescence.

A simple microarray experiment designed to test the effects of ozone on fluorescent dye integrity was designed as follows. First, 20 ug total mouse RNA was reverse transcribed into cDNA in a conventional manner known to those skilled in the art. One such example is described in the Genisphere Submicro Oligo Appendix B protocol, incorporated by reference. Complementary (copy) DNA, cDNA, was prepared using both the Cy3 and Cy5 RT Primer which provided 5 pmole/ul primers. The reverse transcription reactions were stopped as described to yield a final volume of 35 ul. A master microarray hybridization mix containing 10 ug of cDNA in 120 ul of Genisphere's vial 6 hybridization buffer was prepared by combining 17.5 ul each of Cy3 and Cy5 primed cDNA and 12.5 ul each of Cy3 and Cy5 3DNA® Capture reagents (vial 1) (Genisphere, Inc., Montvale, N.J.) with 60 ul of vial 6 hybridization buffer form the Genisphere Submicro Oligo kit. Further details related to the method described above may be found in U.S. patent application Ser. No. 09/802,162, the content of which is incorporated herein by reference.

The resulting master hybridization mixture was heated to 80° C. for 10 minutes in a heat block to denature the cDNA. The mixture was allowed to cool to 55° C. for 45 minutes to prehybridize the cDNA to the 3DNA reagents. After 45 minutes at 55° C., 5 ul of capture sequence blocker were added to the master hybridization mixture and the mixture was incubated at 55° C. for 20 minutes. 25 ul of the mixture was applied to 4 individual microarrays containing 70 mer oligonucleotides generated from known mouse genes. A glass coverslip (22×30) was applied to each microarray and the microarrays were inserted into 50 ml Corning centrifuge tubes arranged horizontally to which 200 ul of deionized water was added. The microarrays were incubated overnight at 55° C. in a dry hybridization oven. On the following morning the four microarrays were washed as described in the Genisphere Submicro Oligo protocol. After spinning until the four microarrays were dry, two of the four microarrays were sprayed with an aerosol for forming the protective coating and two were left untreated. The four microarrays were then scanned in an Axon 4000B microarray scanner. The data from these scans were saved for future comparison. After the data was collected, the four microarrays were placed in a box containing an ozone generator set at maximum. The box was closed and the generator plugged in and turned on. After 5 minutes the ozone generator was turned off and the microarrays were rescanned at the same settings.

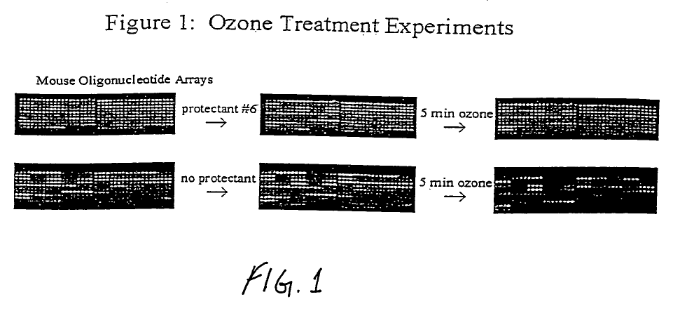

FIG. 1 represents an image of one of the two microarrays treated with the aerosol spray as described and a second untreated microarray. As shown in FIG. 1 the microarray that was sprayed looked nearly identical to the untreated microarray indicating that the protective coating formed from the spray does not interfere with the fluorescent properties of the fluorescent dyes nor does it yield a high background upon application. FIG. 1 also demonstrates that the application of the spray for forming the protective coating clearly prevented degradation of the fluorescent signal of the ‘red’ dye. This was observable by the change of the yellow signal to green and the complete loss of red, Cy5 labeled features on the microarray.

The protective coating may be formed from any material, which possesses the desired properties as previously described. Typical materials for forming the protective coating include polymeric and/or resin materials. In the described embodiment, the protective coating was formed from an acrylic based material and specifically, Crystal Clear Acrylic Coating (Krylon) which included the following ingredients: 2-propanone; dimethylbenzene; petroleum distillates; methylbenzene; naphthalene; 1,2,4-trimethylbenzene; and hydrocarbon propellants.

While the described ingredients provide a successful outcome, the ingredients should not be considered either limiting or required for forming the protective coating that may be used in this application. Components with similar physical or chemical properties when combined with either some or all of the above ingredients or with different ingredients in different quantity or ratios may also fit within the scope of this application. Preferably at least one of the components forming the protective coating is gas impervious which is particularly desirable to protect the label from attack by gases typically found in air (e.g. ozone).

In a still further embodiment of the invention, the protective coating itself or the secondary substrate having the protective coating thereon may be removed from the support means having the label or marker associated therewith. In this way, the support means may be reused after the microassay is completed.

### EXAMPLE 2

### Procedure for Applying a Protective Coating on an Array Substrate

This procedure may be carried out all array substrates. After performing the final preparations of the microarray substrate, the assay substrate was dried using any suitable techniques including centrifugation. The assay substrate was placed in a dark container and a protective coating forming composition was prepared containing each of the following components listed in Table 1.

TABLE 1

| | Amount |
|---|---|
| Components | (% v/v) |
| Copolymer of methyl acrylate and ethyl | 8.75 |
| methacrylate | |
| Naphthalene | 0.52 |
| Acetone | 26.35 |
| Toluene | 64.03 |
| Ethyl-3-ethoxypropionate | 0.35 |

The coating composition was prepared by thoroughly mixing the copolymer with acetone and toluene, and thereafter adding and mixing the remaining components to the mixture. The assay substrate was examined for the presence of dust. The assay substrate was blown with compressed gas to remove any dirt and debris that may have been present thereon. The next few steps required the use of a chemical fume hood and a pair of gloves and protective eyewear. 50 mL of the composition of the present invention was placed into a small glass beaker. The assay substrate was immersed for about 5 seconds to 10 seconds in the composition of the present invention. The assay substrate was removed from the composition of the present invention and allowed to dry for about 3 minutes to 5 minutes. The assay substrate was stored in a dark place until ready to scan. The indicating agent was determined to be stable for at least 3 weeks.

### EXAMPLE 3

A simple microarray experiment designed to test the effects of sunlight exposure and ambient atmosphere on fluorescent dye integrity was designed as follows. A pair of microarrays was prepared using similar procedures as described in Example 1. A protective coating forming composition was prepared having the same formulation as the one disclosed in Example 2.

The composition was applied to one of the prepared microarrays in accordance with the procedure described in Example 2 to yield a treated microarray. The remaining microarray was left untreated. The two microarrays were then scanned in an Axon 4000B microarray scanner. The data from these scans were saved for future comparison. The treated and untreated microarrays were exposed to direct sunlight and ambient air for about 3.5 hours. After the exposure, the microarrays were scanned under the scanner to acquire data for comparison with the pre-exposure scan data.

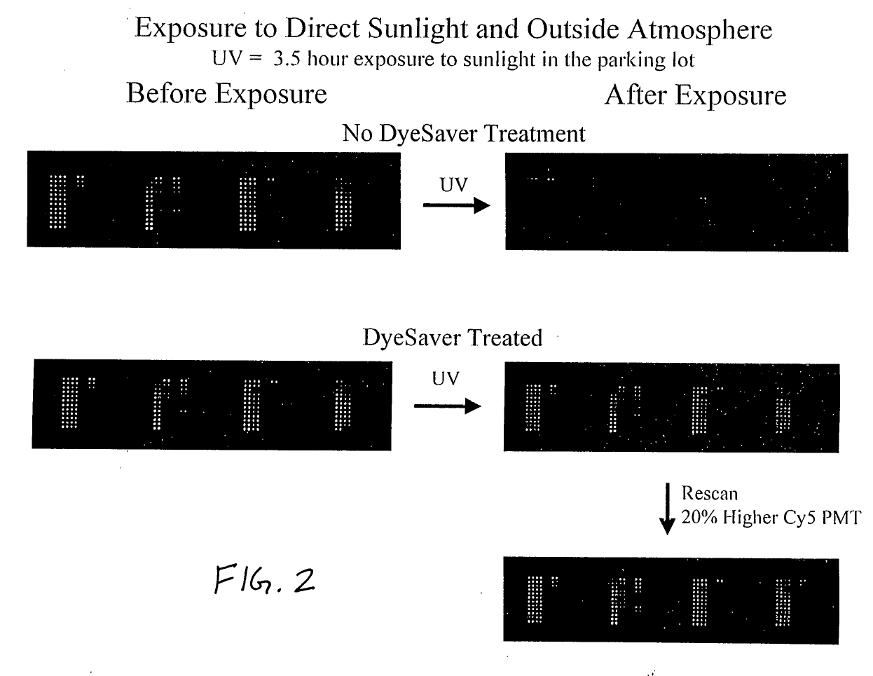

FIG. 2 represents an image of the treated and untreated microarrays prior to and after exposure as described. As shown in FIG. 2, prior to exposure, the microarray that was treated looked nearly identical to the untreated microarray indicating that the protective coating formed from the dip did not interfere with the fluorescent properties of the fluorescent dyes nor did it yield a high background noise upon application. FIG. 2 further demonstrates that the application of the protective coating forming composition for forming the protective coating clearly prevented degradation of the fluorescent signal of the fluorescent dye.

The foregoing discussion discloses and describes merely exemplary embodiments of the present invention. One skilled in the art will readily recognize from such discussion, and from the accompanying drawings and claims, that various changes, modifications and variations can be made therein without departing from the spirit and scope of the invention as defined in the following claims.

What is claimed is:

1. A substrate suitable for use in an assay comprising:
(a) a material for supporting an indicating agent;
(b) an indicating agent capable of generating a detectable signal; and
(c) a protective coating comprised principally of a protective coating forming material at least substantially transparent to the detectable signal generated by the indicating agent.

2. The substrate of claim 1 wherein said protective coating is applied as a composition containing the protective forming material and a delivery system capable of delivering the protective coating forming material to the substrate and evaporating therefrom to leave said protective coating.

3. The substrate of claim 1 wherein the protective coating forming material comprises an acrylic polymer resin.

4. The substrate of claim 2 wherein the acrylic polymer resin is a copolymer of methyl acrylate and ethyl methacrylate.

5. The substrate of claim 1 wherein the protective coating hermetically seals the indicating agent without producing an adverse amount of nonspecific signal generation.

6. The substrate of claim 2 wherein the delivery system comprises at least one evaporable solvent.

7. The substrate of claim 6 wherein the at least one evaporable solvent is selected from the group consisting of water, alcohols, toluene, xylene, heptane, butanol, n-butyl acetate, methyl amyl ketone, ethyl benzene, ethylene glycol butylether, ethyl-3-ethoxypropionate, isopropyl alcohol, ethanol, methoxypropanol, acetone and combinations thereof.

8. The substrate of claim 7 wherein the at least one evaporable solvent is selected from the group consisting of toluene, acetone, ethyl-3-ethoxypropionate and combinations thereof.

9. The substrate of claim 2 wherein the delivery system further comprises a preserving agent for preventing oxidation and other adverse reaction of the protective coating forming material to enhance the transparency of the resulting protective coating during the evaporation of the delivery system.

10. The substrate of claim 9 wherein the preserving agent is an aromatic naphtha.

11. The composition of claim 10 wherein the aromatic naphtha is naphthalene.

12. The composition of claim 1 wherein the protective coating forming material is present in an amount of from about 1% to 90% by volume based on the total volume of the composition.

13. The substrate of claim 12 wherein the protective coating forming material is present in an amount of about 9% by volume.

14. The substrate of claim 9 wherein the preserving agent is present in an amount of from about 0.1% to 10% by volume based on the total volume of the composition.

15. The substrate of claim 14 wherein the preserving agent is present in an amount of 0.52% by volume based on the total volume of the composition.

16. The substrate of claim 2 wherein the composition is in the form selected from the group consisting of a liquid and an aerosol.

17. A composition for forming a protective coating over the surface of an assay substrate having an indicating agent capable of generating a detectable signal associated therewith, comprising a protective coating forming material, and a delivery system for delivering the protective coating forming material in an amount sufficient to coat the surface of the assay substrate, wherein the delivery system evaporates from the surface of the assay substrate to form a protective coating at least substantially composed of the protective coating forming material that is at least substantially transparent to the detectable signal generated by the indicating agent.

18. The composition of claim 17 wherein the protective coating forming material comprises an acrylic polymer resin.

19. The composition of claim 18 wherein the acrylic polymer resin is a copolymer of methyl acrylate and ethyl methacrylate.

20. A method for forming a substantially transparent protective coating over the surface of an assay substrate having an indicating agent capable of generating a detectable signal associated therewith, said method comprising the steps of:
applying to the surface of the assay substrate an effective amount of the composition of claim 17 sufficient to form the protective coating and seal the indicating agent from the ambient atmosphere; and
drying said composition to remove the delivery system to yield the substantially transparent protective coating.

21. The method of claim 20 further comprising polishing the protective coating to enhance the transparent qualities of the protective coating.

22. The method of claim 20 wherein the applying step further comprises:
dipping the assay substrate in the composition of claim 17 for a sufficient time to allow the composition to adhere to the surface of the assay substrate; and
withdrawing the assay substrate from the composition.

23. The method of claim 22 wherein the assay substrate is dipped from about 5 seconds to 10 seconds.

24. The method of claim 20 wherein the applying step further comprises:
pipetting the composition of claim 17 in an amount sufficient to coat the surface of the assay substrate associated with the indicating agent; and
rocking the assay substrate from side to side for a sufficient time to evenly distribute the composition thereacross and form the protective coating.

25. The method of claim 20 wherein the applying step further comprises:
spraying the composition of claim 17 in an amount sufficient to coat the surface of the assay substrate associated with the indicating agent; and
rocking the assay substrate from side to side for a sufficient time to evenly distribute the composition thereacross and form the protective coating.

26. The method of claim 21 wherein the polishing step further comprises:
preparing a polishing solution consisting of a solvent;
suspending the assay substrate in the polishing solution for a sufficient time to remove a layer portion of the protective coating;
withdrawing the assay substrate from the polishing solution;
shaking off any excess polishing solution from the assay substrate;
drying the assay substrate; and
if necessary, repeating the above steps.

27. The method of claim 26 wherein the solvent is selected from the group consisting of toluene, acetone and mixtures thereof.

28. The method of claim 27 wherein the solvent is a mixture containing acetone and toluene in a volumetric ratio of from about 1:2 to 1:3.
